(19) United States
(12) Patent Application Publication
(10) Pub. No.: US 2012/0156713 A1
(43) Pub. Date: Jun. 21, 2012

(54) METHOD FOR ANALYZING AND/OR TREATING A FERTILIZED EGG, AND CORRESPONDING SYSTEM

(76) Inventor: Ephrem Adjanohoun, Pace (FR)

(21) Appl. No.: 13/392,369

(22) PCT Filed: Aug. 25, 2010
(86) PCT No.: PCT/EP2010/062421

§ 371 Date: Feb. 24, 2012

(30) Foreign Application Priority Data
Sep. 3, 2009 (FR) 0904191

(51) Int. Cl. C12N 5/071; C12M 1/00; C12Q 1/02

(52) U.S. Cl. 435/29; 435/375; 435/283.1

(57) ABSTRACT

The present invention relates to a method for analyzing and/or treating a fertilized egg and a system able to implement said method. According to the invention, the method comprises a step of puncturing and/or injecting substance into the albumin sac of the egg, said egg having between 5 and 19 days of incubation and said step of puncturing and/or injecting being achieved by piercing a hole in the shell next to the pointed end of the egg and passing a hollow needle in said hole such as to lead the distal end of said needle into the albumin sac.

The present invention relates to a method for analyzing and/or treating a fertilized egg, and to a system for carrying out said method.

In the poultry industry, particularly in the chick production industry, it is known to treat the eggs in order to cure the chicks from infectious vertical contaminations or to immunize them against infectious diseases and thus reduce their mortality rate. To this end, it is known to immerse the egg entirely in an antibiotic solution. However, this method leads to unsatisfactory results.

It is also known to directly inject a treating substance into the eggs, generally a vaccine and/or antibiotics. The injection is carried out by means of an injector placed over the egg, the flattened end of the egg located near the air space being positioned upwards. A hollow needle is introduced into the egg from its flattened end, crosses the air space of the egg to reach the embryonic fluid and/or the chorioallantoic chambers of the egg. The injection is generally carried out at a late phase of the incubation cycle, at 18 or 19 days in the case of a chicken, or 2 to 3 days before hatching. This method has drawbacks. The injection being carried out at a late stage of the incubation, the duration of the treatment is very short (2 to 3 days). Furthermore, as for the quantities of injected product, they are reduced and limited in general to less than 0.05 milliliters in order to respect the very sensitive osmotic balance of the sacs of embryonic fluid necessary for the development of the embryo. The injectable products are always in liquid form. The injection at the flattened end of the egg may generate bleeding with detrimental consequences on the development of the embryo due to the presence of blood vessels. Finally, the injection of a treating product, in or near the embryonic fluid, often generates bacterial contaminations and may adversely affect the development of the embryo and the hatching of the chick.

The present invention provides a method for analyzing and/or treating a fertilized egg which may be applied at an earlier stage of the incubation cycle and which makes it possible to solve all or part of the aforementioned drawbacks.

More particularly, the present invention relates to a method for analyzing and/or treating a fertilized egg, characterized in that it comprises a step of puncturing and/or injecting a substance into the albumin sac of the egg, said puncturing and/or injecting step being achieved by piercing a hole in the shell near the pointed end of the egg and passing a hollow needle into said hole at an adequate depth so as to lead the distal end of said needle into the albumin sac directly without crossing the embryonic fluid sacs or the vital organs of the embryo and puncturing the substance of the albumin sac and/or injecting a treating substance into the albumin sac.

The puncturing and/or injecting step is carried out on an egg being incubated. The operation can be carried out from the fifth day of incubation and up to 2 days before hatching. In the case of a chicken whose incubation cycle has on average 21 days, the puncturing and/or injecting step is carried out on a fertilized egg having between 5 and 19 days of incubation. At this stage of the incubation cycle of the egg, the albumin sac is formed and positioned near the pointed end of the egg, opposite the air space. The contents of the albumin sac and the membranes crossed by the needle being not or hardly vascularized, the introduction of a needle into this part of the egg does not cause any bleeding and does not disrupt the subsequent development of the egg. Furthermore, at this incubation stage, the substance contained in the albumin sac being hardly humid and exhibiting high compactness, elasticity and viscosity, there is hardly any risk of a contamination occurring and/or bacteria developing therein.

Furthermore, at this incubation stage, the content of the albumin sac is structurally isolated from the rest of the embryo and is only used very slowly by it. Thus, it is possible to take or introduce thereto significant quantities of matter without causing the death of the embryo and preventing imbalances which may harm the immediate or subsequent development of the egg.

In the rest of the description, what is meant by hollow needle is a structure comprising one or several hollow inner ducts making it possible for a substance to pass therethrough.

According to one embodiment, the method only comprises one puncturing step. The puncturing of the substance contained in the albumin sac is for example carried out for the purposes of an analysis in order to characterize the egg or the embryo, for example to determine its development stage or its gender.

According to another particular embodiment, the method only comprises one step of injecting a substance into the albumin sac. The injected substance, called treating substance, is a nutritious or hydrating substance or a diagnostic substance or a therapeutic substance intended to treat pathologies, to stimulate immunity and to boost development of the embryo of the egg or that of the chick after hatching. The injected substance may be in the solid state (soluble or not), or in the liquid state (fluid or pasty), or in the gaseous state or have a mixed consistency or a combination of substances having different states.

According to another particular embodiment, puncturing a substance from the albumin sac is carried out to make room in the albumin sac and then inject, as a replacement, a treating substance. The method thus comprises a step of puncturing a substance into the albumin sac of the egg followed by a step of injecting a treating substance different from the substance punctured from the albumin sac. This embodiment makes it possible to inject more important quantities of treating substance into the egg.

According to a particular application, the treating substance injected into the egg is a nutritious substance intended to promote the growth of the embryo then of the chick after hatching. During the last days of incubation, the contents of the albumin sac are transferred to the yolk sac, which, after the birth of the chick, is found in the chick's abdomen. The nutritious substance is thus used by the chick during its first few days of existence.

According to the invention, the quantity of substance punctured or injected after puncturing ranges between 0.06 milliliters and 3 milliliters, preferably between 0.5 milliliters and 2 milliliters, without any harmful effect on the subsequent development of the embryo and on the hatching of the chick.

According to a particular embodiment, the quantity of injected treating substance is substantially equal to the quantity of punctured substance. However, considering the presence of the air space, these two quantities are not necessarily equal. The size of the air space may vary in order to compensate the difference between the punctured quantity and the injected quantity.

In order to reduce the number of manipulations during the steps of puncturing and injecting, the same hollow needle is used to tap the substance of the albumin sac and inject the treating substance, and puncturing and injecting the substance are carried out through the same hole in the egg so as not to remove the needle from the albumin sac between the puncturing step and the injecting step.

The invention also relates to a system for treating a fertilized egg able to carry out the method and comprising at least a puncturing and injecting device, device which comprises:
a needle, comprising at least an inner duct, able to cross said egg in order to reach the albumin sac of said egg;
syringe type puncturing means, able to communicate with the inner duct of said needle, in order to tap through said needle the substance into the albumin sac of the egg; and
injecting means, different from said puncturing means and able to communicate with the inner duct of said needle, to inject a treating substance through said needle into the albumin sac of the egg, said treating substance at least partially substituting the punctured substance.

According to a particular embodiment, the puncturing means comprise a first tubular body and a first piston slidingly mounted in the first tubular body such as to define a first variable volume chamber for receiving the punctured substance from the albumin sac of the egg, said first tubular body being provided with an inlet orifice opening into said first chamber and able to communicate with the inner duct of the needle and an outlet orifice to discharge the punctured substance out of said first chamber, by moving said first piston, and the injection means comprise a second tubular body and a second piston slidingly mounted in the second tubular body such as to define a second variable volume chamber for storing the treating substance to be injected, said second tubular body being provided with an outlet orifice able to communicate with the inner duct of the needle to discharge the treating substance stored in said second chamber by moving the second piston towards the needle.

According to a particular embodiment, the puncturing means comprise suction means able to create a depression in said first chamber when the first piston is in a spaced position in which it is spaced apart by a non-null predetermined distance from the wall of the distal end of the first tubular body.

Alternatively, the depression is created by moving said first piston between a abutment position wherein said first piston abuts against the wall of the distal end of the first tubular body and said spaced position.

According to a particular embodiment, the second tubular body is mounted on the first piston and the outlet orifice of said second tubular body is provided in said first piston such as to obtain a relatively compact device.

According to a particular embodiment, the outlet orifice of the second tubular body is positioned on the first piston so as to face the inlet orifice of the first tubular body when the first piston is in abutment position.

According to a particular embodiment, the outlet orifice of the second tubular body comprises at least a hole having a reduced section for preventing any passage of substance from the first chamber to the second chamber. Alternatively, the outlet orifice of the second tubular body has a larger section and is provided with a perforated gate with holes having a reduced section for preventing any passage of substance from the first chamber to the second chamber.

According to a particular embodiment, the system according to the invention further comprises moving means able to move the puncturing and injecting device between a rest position in which the needle is outside the egg to be treated and an active puncturing and/or injecting position, called active position, wherein the distal end of the needle is in the albumin sac of the egg to be treated.

The invention will be better understood, and other purposes, details, characteristics and advantages will become more apparent from the following detailed explicative description of the two currently-preferred particular embodiments of the invention, with reference to the accompanying schematic drawing, wherein:

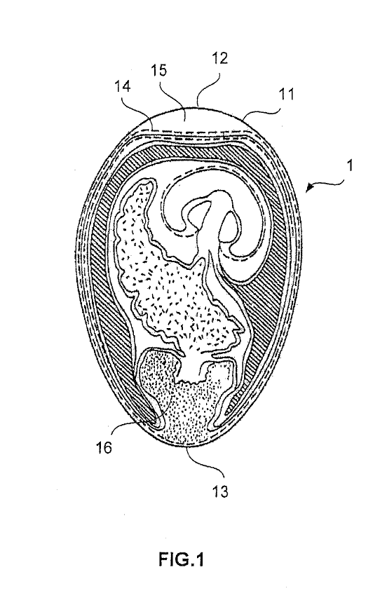

FIG. 1 is a longitudinal cross-section of a fertilized egg;

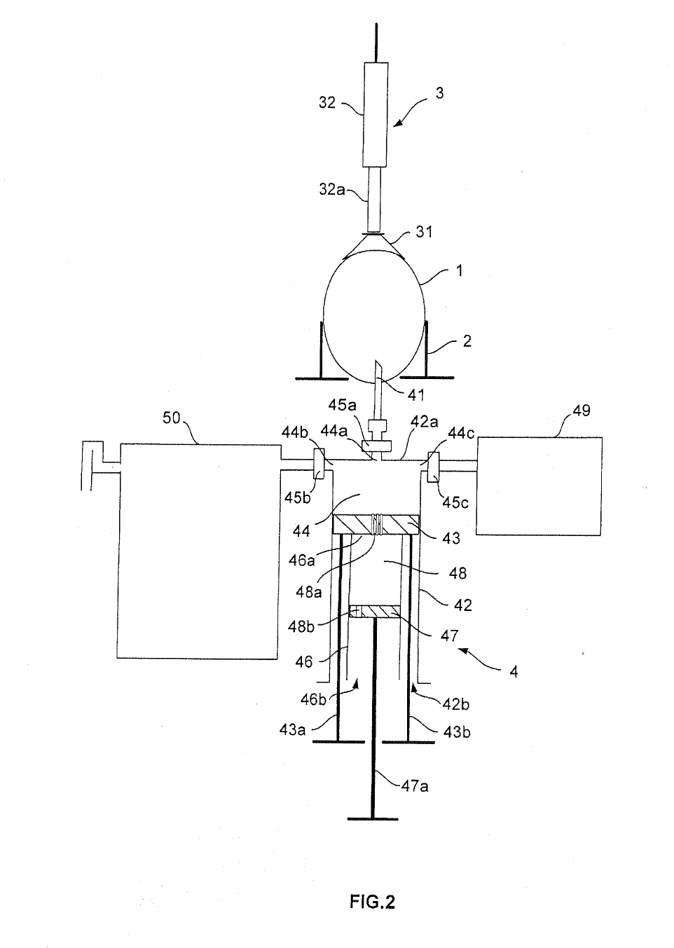

FIG. 2 represents a device able to carry out the method of the invention; and,

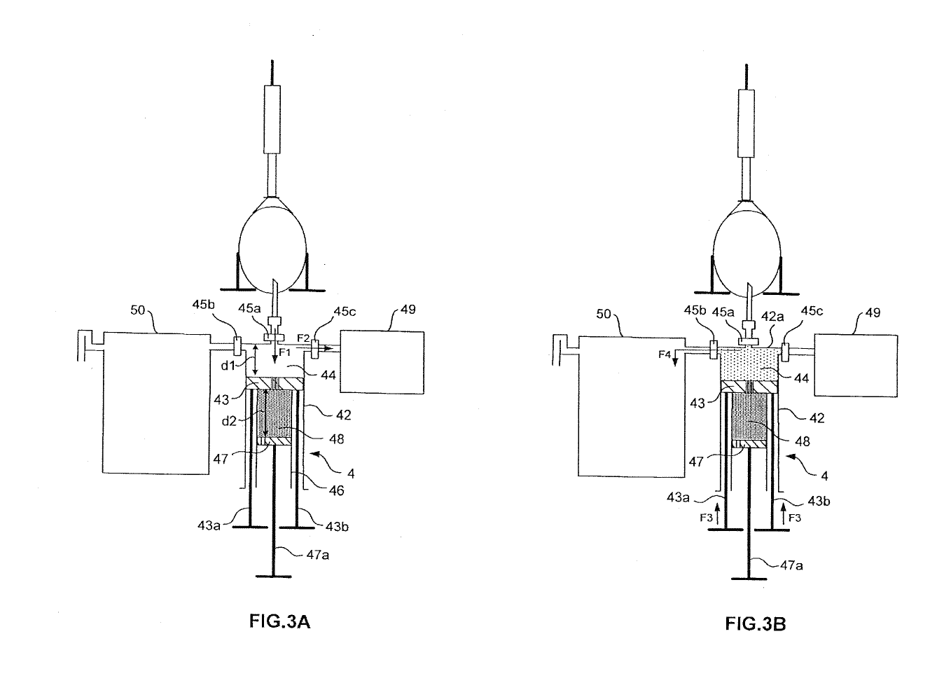

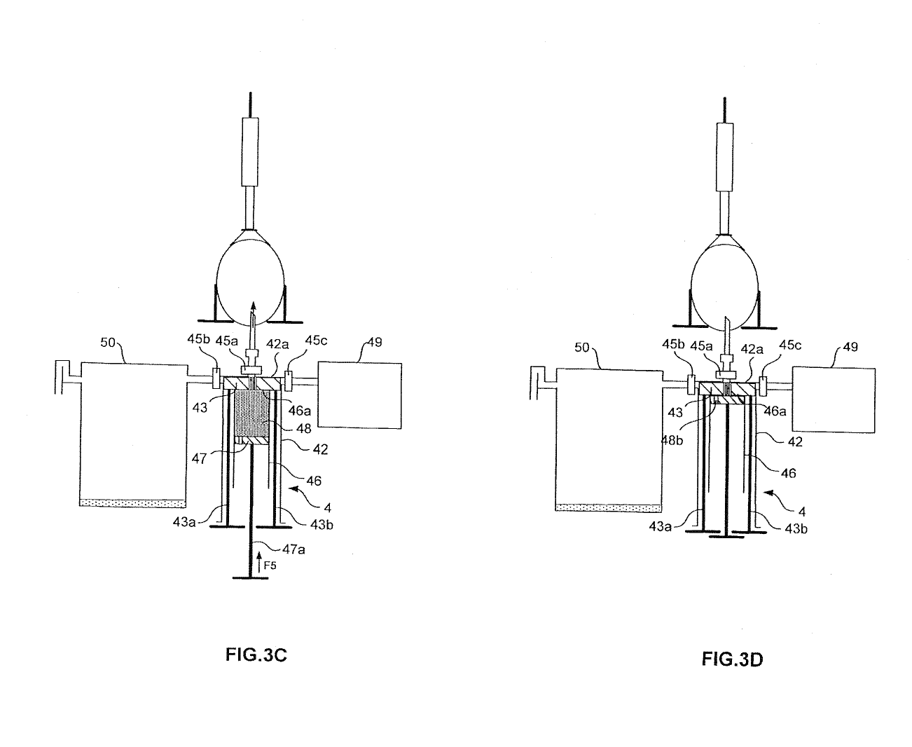

FIGS. 3A to 3D illustrate the steps of the method of the invention when it is carried out by the device of FIG. 2.

The method is applied on chicken eggs having incubated for at least 5 days and is applied at the most two days before the end of the incubation cycle (which corresponds to the hatching of the embryo of the fertilized egg), that is to say between 5 and 19 days of incubation in the case of a chicken egg. At this incubation stage, the albumin sac is already formed and positioned in the lower portion of the egg and the contents of the albumin sac are structurally isolated from the rest of the embryo and are only very gradually used by the embryo. The contents of the albumin sac are only used significantly by the embryo at a later stage of development of the embryo, right at the end of incubation. Thus, it is possible to take a sample or introduce significant quantities of matter without causing osmotic shocks or the death of the embryo and by avoiding imbalances which may harm the immediate or later development of the egg.

FIG. 1 is a longitudinal cross-section of a fertilized egg 1 having around 14 days of incubation. This egg comprises a shell 11 of ovoid shape exhibiting a flattened end 12 and a pointed end 13. The inside of the shell 11 is lined with a very fine outer shell membrane (not represented) and an inner shell membrane 14. The egg exhibits, at its flattened end 12, an air space 15 formed between the outer shell membrane and inner shell membrane, and, near its pointed end 13, an albumin sac 16.

According to the invention, a puncturing of substance and/or an injection of treating substance is carried out in the albumin sac 16 by piercing a hole in the shell at the pointed end of the egg and the passing of a hollow needle in the hole such as to lead the distal end of the needle into the albumin sac. Piercing a hole in the shell may be carried out with the needle or by any other means.

In the rest of the description, is described the case where puncturing the substance into the albumin sac is first carried out and then a treating substance is injected in place of the punctured substance, for example a nutritious liquid substance intended to boost the development of the chick after hatching.

The quantity of punctured and/or injected substance ranges between 0.06 ml and 3 ml, preferably between 0.5 ml and 2 ml. Advantageously, the quantity of injected substance is substantially equal to the quantity of punctured substance. The puncturing may be carried out in one or several steps. The same applies for injecting.

The puncturing is carried out by means of a hollow needle which is introduced into the egg at its pointed end 13. The albumin sac as well as the areas traversed by the needle particularly the inner and outer shell membranes, being non vascularized areas of the egg, there is no risk of bleeding.

The needle is introduced into the egg over a length ranging between 0.1 cm and 2.5 cm, preferably between 0.3 cm and 1.5 cm to reach the albumin sac.

The substance contained in the albumin sac 16 is punctured by creating a strong depression, for example by means of a vacuum pump, in the needle and the chamber intended to receive the substance in order to break the molecular bonds of the substance and to only tap one portion of the albumin sac contents.

The same needle is then used to inject the treating substance into the egg. Thus, the needle need not be removed from the egg after the puncturing operation.

A block diagram of a system able to carry out the method of the invention is represented on FIG. 2.

The system comprises a support 2 whereon the egg 1 to treat is arranged, a vertical blocking device 3 arranged above the egg to be treated in order to block it in the support 2 and a puncturing and injecting device 4 for puncturing and injecting the substance into the egg.

The support 2 is for example an incubating tray provided with eyelets underneath and comprising a plurality of cells arranged in rows and columns and intended to each receive one egg. The egg to be treated is arranged in a cell such that its pointed end is accessible from beneath the tray. The egg is maintained stationary in the cell by means of the cell walls and the vertical blocking device 3. The vertical blocking device 3 is for example formed of a supporting cup 31, in flexible material, mounted at the end of the stem 32a of a jack 32. The jack 32 is able to move the stem 32a between a top position and a bottom position. In the bottom position, the supporting cup 31 presses against the flattened end of the egg and prevents any vertical movement of the egg in the cell.

The puncturing and injecting device 4 is positioned beneath the support 2. Generally, it comprises a needle 41 comprising an inner duct which communicates with puncturing means of syringe type and injecting means.

The puncturing means comprise a substantially cylindrical tubular body 42 with a closed distal end 42a and an open proximal end 42b. A piston 43 is slidingly mounted in the tubular body 42 such as to define a first variable volume chamber 44 intended to receive the punctured substance from the egg. The piston 43 is connected to two stems of the piston 43a and 43b arranged substantially symmetrically with respect to the centre of the piston and extending beyond the proximal end 42b. The two stems of the piston serve to move the piston in the chamber 44 between a spaced position wherein said piston is spaced apart by a non null distance from the distal end 42a and an abutment position where it abuts against the wall of the distal end 42a. The free end of the two stems of the piston possibly cooperates with push-type activating means, not represented, able to move the piston in the chamber.

The tubular body 42 is furthermore provided at its distal end 42a with an inlet orifice 44a opening into the chamber 44 and able to communicate with the inner duct of the needle 41. It is also provided on its lateral wall near the distal end wall 42a, with an outlet orifice 44b to discharge from the chamber, the punctured substance contained in the chamber 44 and evacuate it towards a recipient 50 connected to the outlet orifice 44b. Alternatively, the outlet orifice 44b is positioned on the distal end 42a beside the inlet orifice 44a.

Suction means, such as a vacuum pump 49, are provided to create a depression in the chamber 44 when the piston 43 is in the spaced position. These suction means are connected to an orifice 44c of the tubular body opening into the chamber 44.

Orifices 44a, 44b and 44c are provided with solenoid valves 45a, 45b and 45c controlled to open and close by a control circuit not represented on FIG. 2. The driving of the solenoid valves will be described further on in the description with reference to FIGS. 3A to 3D illustrating the operation of the system.

In the present embodiment, the injection means are mounted on the piston 43 for reasons of compactness. The injecting means comprise a tubular body 46, mounted on the piston 43 between the two piston stems 43a and 43b, having a closed distal end 46a corresponding to the lower wall of the piston and an open proximal end 46b. A piston 47 is slidingly mounted in the tubular body 46 such as to define a variable volume chamber 48 for storing the treating substance to be injected. The piston 47 is connected to a piston stem 47a extending beyond the proximal end 46b. The piston stem aims to move the piston 47 in the chamber 48 between a spaced position wherein it is spaced at a non null distal from the distal end 46a and an abutment position where it abuts against the wall of the distal end 46a. The free end of the piston stem possibly cooperates with push-type activating means, not represented, able to move the piston in the chamber 48.

The chamber 48 is sized so as to receive one or several doses of treating substance.

An outlet orifice 48a is provided in the piston 43 to evacuate, by moving the piston 47 towards the distal end 48a, the treating substance contained in the chamber 48 towards the inner duct of the needle when the piston 43 is in the abutment position. In the embodiment, the outlet orifice 48a is composed of a plurality of holes of small cross-section, preventing the substance present in the chamber 44 to pass into the chamber 48. The orifice 48a is positioned to be faced with the orifice 45a when the piston 43 is in abutment against the wall of the distal end of the tubular body 42. An orifice 48b intended to supply the chamber 48 with the treating substance is also provided in the piston 47. This orifice is closed apart during the filling phases of the chamber 48.

The system also comprises moving means, not represented on the figures, able to move the puncturing and injecting device 4 between a rest position wherein the needle is outside the egg to be treated and an active puncturing and/or injection position, called active position, wherein the distal end of the needle is in the albumin sac of the egg to be treated.

A description of a puncturing and injection cycle by means of the treating system of the invention will now be carried out with reference to FIGS. 3A to 3D. In all these figures, the moving means of the puncturing and injecting device are considered to be in the active position such that the distal end of the needle 41 is positioned in the albumin sac of the egg. It is also considered that when the piston 47 is in the spaced position, the volume of the chamber 48 corresponds to a dose of treating substance to inject in the egg.

FIG. 3A illustrates the start of the cycle. The pistons 43 and 47 are in a spaced position, the solenoid valves 45a and 45c are in an open position and the solenoid valve 45b is in a closed position. The chamber 48 encloses a dose of treating substance to inject.

The vacuum pump 49 creates a depression in the chamber 44 such that a predetermined quantity of substance from the albumin sac of the egg falls into the chamber 44 following arrow F1. The moving of the air suctioned by the vacuum pump is represented by the arrow F2. The volume V1 of the chamber 44 determines the quantity of substance to extract from the albumin sac. This volume is defined by the size of the section of the tubular body 42 and the distance d1 between the piston 43 and the distal end 42a. Likewise, the volume V2 of the chamber 48, which corresponds in the present embodiment to a dose of treating substance to inject, is defined by the size of the section of the tubular body 42 and the distance d1 between the piston 43 and the distal end 42a.

A quantity of substance corresponding to the volume V1 is discharged into the chamber 44 by gravity and under the effect of the depression created by the vacuum pump. The depression is created in the chamber 44 at the start of the puncturing phase. The solenoid valves 45a and 45c are open at the start of this phase then the solenoid valve 45c is closed as soon as a sufficient depression is created in the chamber 44. According to an alternative embodiment, the depression is created in the chamber 44 before the actual puncturing phase.

FIG. 3B illustrates the end of the puncturing phase. A volume V1 of substance has been taken from the albumin sac of the egg and is now present in the chamber 44.

The following phase is a phase of emptying the chamber 44 by opening the solenoid valve 45b and by pushing the piston stems 43a and 43b towards the distal end 42a, as indicated by the arrows F3. The piston 43 slides in the chamber 44 until it abuts against the distal end 42a. During this phase, the solenoid valves 45a and 45c are closed and the content of the chamber 44 is discharged into the recipient 50 as indicated by arrow F4. At the end of this phase, the piston 43 abuts against the distal end 42a and the punctured substance is present in the recipient 50, as illustrated by FIG. 3C. The outlet orifice 48a is facing the orifice 44a. Injecting the treating substance contained in the chamber 48 may thus be carried out by pushing the piston stem 47a towards the distal end 46a, as indicated by arrow F5. The piston stem is pushed to supply a dose of treating substance. In the example described here, the chamber 48 being sized to receive one single dose of treating substance, the piston 47a stem is thus pushed until the piston 47 abuts against the distal end 46a. FIG. 3D illustrates the end of this phase. The piston 43 abuts against the distal end 42a of the tubular body 42 and the piston 47 abuts against the distal end 46a of the tubular body 46.

Before starting a puncturing and injecting cycle again, the pistons 43 and 47 are brought to spaced position and the chamber 48 is filled with treating substance from the orifice 48b.

Of course, in the case where the chamber 48 is sized to receive several doses of treating substance, the piston 47 is only brought into the spaced position when all the doses have been injected (the piston is thus in abutment with the distal end 46a.)

One may thus treat an egg without having to remove the needle from the egg. Furthermore, after extracting the needle from the egg, it is not necessary to seal the hole present in the shell considering that the compact nature of the punctured substance. Sealing the hole may nevertheless be achieved with wax or with other adapted components.

Although the invention has been described in connection with different particular embodiments, it is obvious that it is in no way limited thereto and that it encompasses all the technical equivalents of the means described as well as their combinations should these fall within the scope of the invention.

1-16. (canceled)

17. A method of analyzing and/or treating a fertilized egg, characterized in that it comprises a step for puncturing and/or injecting a substance into the albumin sac of the egg, said puncturing and/or injecting step being carried out by piercing a hole in the shell next to the pointed end of the egg and passing a hollow needle within said hole such as to lead the distal end of said needle into the albumin sac
and in that the puncturing and/or injecting step is carried out on an incubated egg having at least 5 days of incubation, and at least 2 days before hatching.

18. The method according to claim 17, characterized in that it comprises a step of puncturing a substance from the albumin sac of the egg followed by a step of injecting a treating substance.

19. The method according to claim 18, characterized in that the substance is different from the substance punctured in the albumin sac.

20. The method according to claim 18, characterized in that the quantity of substance punctured or injected after puncturing ranges between 0.06 milliliters and 3 milliliters, preferably between 0.5 milliliters and 2 milliliters.

21. The method according to claim 18, characterized in that the injected treating substance is liquid, solid or gaseous.

22. The method according to claim 18, characterized in that the quantity of injected treating substance is substantially equal to the quantity of punctured substance.

23. The method according to claim 18, characterized in that the same hollow needle is used to puncture the substance from the albumin sac and inject the treating substance.

24. The method according to claim 23, characterized in that the puncturing and injecting of the substance are carried out through the same hole.

25. The method according to claim 18, characterized in that said treating substance is a nutritious substance, a diagnostic substance or a therapeutic substance.

26. A system for treating a fertilized egg, characterized in that it comprises at least a puncturing and injecting device (4) comprising:
a needle (41), comprising at least an inner duct, able to traverse said egg to reach the albumin sac of said egg;
puncturing means (42, 43, 44, 49), able to communicate with the inner duct of said needle, to puncture through said needle substance from the albumin sac of the egg; and
injecting means (46, 47, 48), different from said puncturing means and able to communicate with the inner duct of said needle, to inject through said needle a treating substance into the albumin sac of the egg, said treating substance at least partially substituting the punctured substance.

27. The system according to claim 26, characterized in that the puncturing means comprise a first tubular body (42) and a first piston (43) slidingly mounted in the first tubular body such as to define a first chamber (44) for receiving the punctured substance from the albumin sac of the egg, said first tubular body being provided with an inlet orifice (44a) opening into said first chamber able to communicate with the inner duct of the needle and an outlet orifice (44b) to discharge the punctured substance from said first chamber by moving said first piston,
and in that the injection means comprise a second tubular body (46) and a second piston (47) slidingly mounted in the second tubular body such as to define a second chamber (48) for storing the treating substance to be injected, said second tubular body being provided with an outlet orifice (48a) able to communicate with the inner duct of the needle to discharge the treating substance stored in said second chamber towards the needle by moving the second piston.

28. The system according to claim 27, characterized in that the puncturing means comprise suction means (49) able to create a depression in said first chamber when the first piston is in a spaced position.

29. The system according to claim 27, characterized in that the second tubular body (46) is mounted on the first piston (43) and in that the outlet orifice (48a) of said second tubular body is provided in said first piston.

30. The system according to claim 29, characterized in that the outlet orifice (48a) of the second tubular body comprises at least a hole having a cross-section for preventing any passage of punctured substance from said first chamber to said second chamber.

31. The system according to claim 26, characterized in that it further comprises moving means able to move the puncturing and injecting device between a rest position wherein the needle is outside the egg to be treated and an active puncturing and/or injection position, called active position, wherein the distal end of the needle is in the albumin sac of the egg to be treated.

32. The system according to claim 28, characterized in that the second tubular body (46) is mounted on the first piston (43) and in that the outlet orifice (48a) of said second tubular body is provided in said first piston.

33. The system according to claim 32, characterized in that the outlet orifice (48a) of the second tubular body comprises at least a hole having a cross-section for preventing any passage of punctured substance from said first chamber to said second chamber.
